(19) United States
(12) Patent Application Publication
(10) Pub. No.: US 2026/0145000 A1
(43) Pub. Date: May 28, 2026

(54) HYPOTHERMIA APPLICATOR FOR HUMAN BODY TEMPERATURE RISE

(71) Applicant: KING FAHD UNIVERSITY OF PETROLEUM AND MINERALS, Dhahran (SA)

(72) Inventors: Rifaqat HUSSAIN, Dhahran (SA); Abdul Rehman CHISHTI, Bahawalpur (PK); Abdul AZIZ, Bahawalpur (PK); Abdullah AL-GARNI, Dhahran (SA)

(73) Assignee: KING FAHD UNIVERSITY OF PETROLEUM AND MINERALS, Dhahran (SA)

(21) Appl. No.: 19/377,097

(22) Filed: Nov. 3, 2025

Related U.S. Application Data
(62) Division of application No. 18/452,856, filed on Aug. 21, 2023.
(60) Provisional application No. 63/446,176, filed on Feb. 16, 2023.

(51) Int. Cl. A61N 5/02; H01Q 9/04; H01Q 9/26; H05B 6/80

(52) U.S. Cl. CPC A61N 5/025; H01Q 9/0414; H01Q 9/26; H05B 6/80

(57) ABSTRACT

A folded dipole patch antenna for use in a hyperthermia applicator is described. The antenna includes a dielectric circuit board, and a folded dipole microstrip antenna. The dielectric circuit board has a top side and a bottom side. The folded dipole microstrip antenna is formed on the top side and includes two meander paths mirror each other. A pair of parallel metallic strips is located on the bottom side. A coaxial feed port is connected to the pair of parallel metallic strips. The antenna is configured to resonate in a frequency range of about 434 MHz upon application of an input signal at the coaxial feed port. The folded dipole patch antenna has a small size and does not require an impedance matching circuit. The hyperthermia applicator is configured to emit microwave energy toward a target tissue within a human body.

### CROSS REFERENCE TO RELATED APPLICATIONS

The present application claims the benefit of priority to U.S. Prov. App. No. 63/446,176 entitled “Miniaturized Folded Dipole Patch Antenna for Sub-1 Ghz Band Applications”, filed on Feb. 16, 2023, and incorporated herein by reference in its entirety.

### STATEMENT REGARDING PRIOR DISCLOSURE BY THE INVENTORS

Aspects of this technology are described in U.S. application Ser. No. 18/183,796, filed on Mar. 14, 2023, which is incorporated herein by reference in its entirety.

### BACKGROUND

### Technical Field

The present disclosure is directed to a miniaturized folded dipole patch antenna for sub-GHz band applications.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Antennas play an important role in wireless communication, especially in low-power sensing and monitoring applications, biomedical applications, and Internet of Things (IoT) devices. Antennas have been increasingly used in biomedical applications. For example, antenna sensors have been used to monitor various parameters associated with the human body, such as temperature, glucose monitoring, heart rate, and the like. In addition, biomedical antennas are used for cardiac pacemakers, endoscopy, and cancer treatment, and hyperthermia treatments. Hyperthermia treatments use a biomedical antenna, which applies a defined temperature to a cancer patient's skin for a limited duration to destroy cancer cells. Biomedical antennas operating in low-frequency bands are more suitable for cancer treatment by hyperthermia due to the higher penetration depth of electromagnetic fields at low frequencies.

To operate a biomedical antenna in a low-frequency bands, a frequency shifting technique has been described, where multiple lumped elements are added to a folded dipole. Increasing inductance of the antenna shifts the resonance below a desired resonance frequency, while a smaller capacitance value shifts the reflection coefficient curve towards the resonance frequency. (See: Das, Sanghamitro, et al., “A strongly miniaturized and inherently matched folded dipole antenna for narrowband applications,” published in IEEE Transactions on Antennas and Propagation 68.5, 2020; pp. 3377-3386). However, a large antenna requires complex parametric analysis of lumped elements and a balun transformation to achieve a desired impedance match.

In biomedical applications, particularly in implants, medical devices are required to be of smaller size, as small devices can reduce a possibility of rejection of implants by the body and can alleviate pain of patients. The design of such medical devices includes parameters such as miniaturization, biocompatibility, patient safety, improvement in communication quality, and the like. Miniaturization is required to integrate the biomedical antenna into small medical devices. Therefore, a challenge in designing a compact antenna for low-frequency bands is to miniaturize its size yet retain suitable radiation performance. Also, a small antenna size requires a complex matching network for impedance matching because of its small input resistance and a large reactance at the input terminal. The performance of an antenna used in hyperthermia treatments is also influenced by its proximity to the human body. Therefore, the design of a miniaturized antenna with stable radiation performance near the human body is a difficult task.

A folded dipole feed structure where an electrically small antenna has a coplanar stripline (CPS) and capacitively loaded loops (CLL) to achieve a quasi-isotropic radiation pattern has been described. The antenna dimensions were defined as 20.6 mm×20.4 mm×0.787 mm. The antenna is fed through a coaxial feed applied at the CPS. (See: J. Ouyang, Y. M. Pan, S. Y. Zheng and P. F. Hu, “An Electrically Small Planar Quasi-Isotropic Antenna,” in IEEE, Antennas And Wireless Propagation Letters, Vol. 17, No. 2, pp. 303-306, February 2018). However, this antenna is large and resonates at 2.4 GHz.

Further, a conformal and flexible slot microstrip antenna that uses multiple layers of silicon to improve SAR centralization for hyperthermia applications has been described (see: Rajebi, Saman, et al., “SAR enhancement of slot microstrip antenna by using silicon layer in hyperthermia applications,” published in Wireless Personal Communications, Vol.

111.3). However, the antenna encompasses a volume of 1.93 mm×124 mm×124 mm, as the addition of multiple layers of silicon significantly increases complexity and bulk in the antenna design. Hyperthermia applicators can use the Industrial Scientific and Medical (ISM) frequency bands of about 27 MHz, about 434 MHz, about 915 MHz and about 1.45 GHz. The longer wavelength of 434 MHz has shown a more uniform specific absorption rate (SAR) and greater penetration depth than the frequencies of 915 MHz and 2.45 GHz (see Curto, Sergio, “Antenna Development for Radio Frequency Hyperthermia Applications”, June 2010, thesis, Technical University Dublin, Dublin, Ireland. However, the antenna of this reference is of large size, having a surface area of 100×100 mm².

Conventional antennas used in hyperthermia treatments are not miniaturized. Hence, there is a need for a folded dipole patch antenna that is configured to operate in sub-GHz frequencies for biomedical applications, has a small size, and requires no specific devices for impedance matching.

### SUMMARY

In an embodiment, a folded dipole patch antenna is described. The folded dipole patch antenna includes a dielectric circuit board, a folded dipole microstrip antenna, a first gap, a lumped inductor, a second gap, a pair of parallel metallic strips, a third gap, and a coaxial feed port. The dielectric circuit board includes a top side, a bottom side, a first edge, a second edge parallel to the first edge, a third edge perpendicular to the first edge and the second edge, a fourth edge parallel to the third edge, a first central axis which extends from the first edge to the second edge, and a second central axis which extends from the third edge to the fourth edge. The folded dipole microstrip antenna is formed on the top side. The folded dipole microstrip antenna includes two meander paths, each having mirror geometry about the second central axis. The first gap is centered on the second central axis between the two meander paths and near the third edge. The lumped inductor is inserted across the first gap near the third edge. The second gap is centered on the second central axis between the two meander paths near the fourth edge. The pair of parallel metallic strips is located on the bottom side. The pair of parallel metallic strips extends from the fourth edge towards the third edge. The pair of parallel metallic strips has mirror geometry about the second axis. A third gap is located between the pair of parallel metallic strips. A coaxial feed port is connected to the pair of parallel metallic strips at the fourth edge. The folded dipole patch antenna is configured to resonate in a frequency range of about 434 MHz upon application of an input signal at the coaxial feed port.

In another exemplary embodiment, a hyperthermia applicator for use in hyperthermia medical treatments to induce a temperature rise in a target area of a human body is described. The hyperthermia applicator includes a dielectric circuit board, a folded dipole microstrip antenna, a first gap, a lumped inductor, a second gap, a pair of parallel metallic strips, a third gap, a power supply, a signal generator, a coaxial cable, and a coaxial feed port. The dielectric circuit board has a top side, a bottom side, a first edge, a second edge parallel to the first edge, a third edge perpendicular to the first edge and the second edge, a fourth edge parallel to the third edge, a first central axis which extends from the first edge to the second edge, and a second central axis which extends from the third edge to the fourth edge. The folded dipole microstrip antenna is formed on the top side. The folded dipole microstrip antenna includes two meander paths, each having mirror geometry about the second central axis. The first gap is centered on the second central axis between the two meander paths and near the third edge. The lumped inductor inserted across the first gap near the third edge. The second gap is centered on the second central axis between the two meander paths near the fourth edge. The pair of parallel metallic strips is located on the bottom side. The pair of parallel metallic strips extends from the fourth edge towards the third edge. The pair of parallel metallic strips has mirror geometry about the second axis. The third gap is located between the pair of parallel metallic strips. The signal generator is connected to the power supply. The signal generator is configured to generate an alternating voltage in a microwave frequency range. The coaxial cable is connected to the signal generator. The coaxial feed port is connected to the coaxial cable at a receiving end, and is connected to the pair of parallel metallic strips at the fourth edge. The folded dipole patch antenna is configured to resonate in a frequency range of about 434 MHz upon receiving the alternating voltage in the microwave frequency range at the coaxial feed port and emit microwave energy, and wherein the microwave energy is configured to raise the temperature of the target area when the hyperthermia applicator is placed over the target area of the human body.

In another exemplary embodiment, a folded dipole patch antenna is described. The folded dipole patch antenna includes a dielectric circuit board, a folded dipole microstrip antenna, a pair of parallel metallic strips, and a coaxial feed port. The dielectric circuit board includes a top side, a bottom side, a first edge, a second edge parallel to the first edge, a third edge perpendicular to the first edge and the second edge, a fourth edge parallel to the third edge, a first central axis which extends from the first edge to the second edge, and a second central axis which extends from the third edge to the fourth edge. A length of the dielectric circuit board between the first edge and second edge is about 16.4 mm and a width of the dielectric circuit board between the third edge and the fourth edge is about 8.6 mm. The folded dipole microstrip antenna is formed on the top side. The folded dipole microstrip antenna includes five sections. A first section includes a first leg parallel to the third edge, a second leg connected to the first leg and parallel to the first edge, and a third leg connected to the second leg and parallel to the first leg. A second section includes a straight leg parallel to the third edge. The second section has a first end separated from a first end of the first leg of the first section by a first gap. A third section includes a leg parallel to the fourth edge and an arm perpendicular to and connected to the leg. The arm is configured to extend towards the fourth edge. A first end of the leg is separated by a second gap from a second end of the first section. A fourth section includes a leg parallel to the fourth edge and an arm perpendicular to and connected to the leg. The arm is configured to extend towards the fourth edge. The arm of the fourth section is separated from the arm of the third section by a third gap. A fifth section includes a first leg parallel to the third edge, a second leg connected to the first leg and parallel to the second edge, and a third leg connected to the second leg and parallel to the first leg. A first end of the fifth section is separated by a fourth gap from the straight section and a second end of the fifth section is separated by a fifth gap from a first end of the leg of the fourth section. A first inductor is located in the first gap. A first capacitor is located in the second gap. A second inductor is located in the fourth gap. A second capacitor is located in the fifth gap. The pair of parallel metallic strips is located on the bottom side. The pair of parallel metallic strips is configured to extend from the fourth edge towards the third edge. The pair of parallel metallic strips has mirror geometry about the second axis. The coaxial feed port is connected to the pair of parallel metallic strips at the fourth edge. The inductance values of the first inductor and the second inductor and capacitance values of the first capacitor and the second capacitor are selected such that the folded dipole patch antenna resonates at a frequency of about 434 MHz upon application of an input signal at the coaxial feed port.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a top view of a folded dipole patch antenna, according to certain embodiments.

FIG. 1B is a bottom view of the folded dipole patch antenna, according to certain embodiments.

FIG. 2A is a schematic top view of the folded dipole patch antenna, according to certain embodiments.

FIG. 2B is a schematic bottom view of the folded dipole patch antenna, according to certain embodiments.

FIG. 3 is a graph of a reflection coefficient curve having s-parameters (S₁₁) when the antenna is fed at a top side of a dielectric circuit board, according to certain embodiments.

FIG. 4 is a graph of the reflection coefficient curve having s-parameters (S₁₁) when the antenna is fed at a bottom side of the dielectric circuit board, according to certain embodiments.

FIG. 5A is a top view of the folded dipole patch antenna with multiple lumped elements, according to certain embodiments.

FIG. 5B is a graph of the reflection coefficient curve having s-parameters (S₁₁) when the antenna has multiple lumped elements with fixed capacitance and variable inductance, according to certain embodiments.

FIG. 5C is a graph of the reflection coefficient curve having s-parameters (S₁₁) when the antenna has multiple lumped elements with fixed inductance and variable capacitance, according to certain embodiments.

FIG. 6 is a graph representing an effect of variations in inductance on the reflection coefficient curves having s-parameters (S₁₁), according to certain embodiments.

FIG. 7 is a graph representing an effect of variations in an arm length (L₃) on the reflection coefficient curves having s-parameters (S₁₁), according to certain embodiments.

FIG. 8 is a graph representing an effect of variations in an arm width (L₅) on the reflection coefficient curves having s-parameters (S₁₁), according to certain embodiments.

FIG. 9 is a graph representing a simulated reflection coefficient curve of the folded dipole patch antenna, according to certain embodiments.

FIG. 10A is a representation of a measured electric field when the folded dipole patch antenna is placed on a human hand, according to certain embodiments.

FIG. 10B is a representation of a measured specific absorption rate (SAR) field when the folded dipole patch antenna is placed on the human hand, according to certain embodiments.

FIG. 10C is a top view of a hand illustrating an effective field strength when the folded dipole patch antenna is placed on the human hand, according to certain embodiments.

FIG. 11A is a representation of penetration depth of the antenna inside the human hand calculated using the SAR field, according to certain embodiments.

FIG. 11B is a representation of penetration depth of the antenna inside the human hand using an electric field, according to certain embodiments.

FIG. 12 represents a block diagram of a hyperthermia applicator, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

Aspects of the present disclosure are directed to a miniaturized folded dipole patch antenna for biomedical applications. Aspects of the present disclosure describe a folded dipole patch antenna having dimensions of 16.40 mm×8.60 mm (0.023λ×0.012λ). In the antenna, a feeding mechanism with coplanar strips is used for indirect feed coupling, thereby reducing antenna size by about 93%. The antenna employs a single inductor and achieves a resonance notch at 434 MHz, which is suitable for biomedical applications, particularly in hyperthermia procedures which induce heating within a target area of a human body. The resonance frequency of 434 MHz has a uniform specific absorption rate (SAR) and good penetration depth. Variations in the inductor value result in frequency shifting. The antenna is configured to provide impendence matching without using a balun transformer or any other complex matching network. The antenna was installed in a hyperthermia applicator and placed on a human body model to evaluate its performance for hyperthermia procedures by measuring effective field strength (EFS) and penetration depth.

In various aspects of the disclosure, definitions of one or more terms that will be used in the document are provided below.

The term “antenna feed” refers to a connector that connects a transmitter or receiver with an antenna and makes the two devices compatible. The connector may a cable, a conductor, a coaxial cable, a twin-lead, a ladder line or a waveguide.

The term “decibel (or dB)” is a unit used to measure the ratio of input to output power. dB measures the intensity of the power level of an electrical signal by comparing it to a given scale. For example, an amplifier causes a gain in power measured in decibels and it is indicated by a positive number. In another example, cables can cause a loss of power. This is measured in negative dB.

The term “folded” with respect to antennas means that the antenna structure curves to form a closed shape. For example, a folded dipole antenna is a half-wave dipole antenna with an additional parallel wire or rod connecting its two ends and folded to form a cylindrical closed shape.

The term “specific absorption rate (SAR)” is a measure of the rate at which energy is absorbed per unit mass by a human body when exposed to a radio frequency (RF) electromagnetic field.

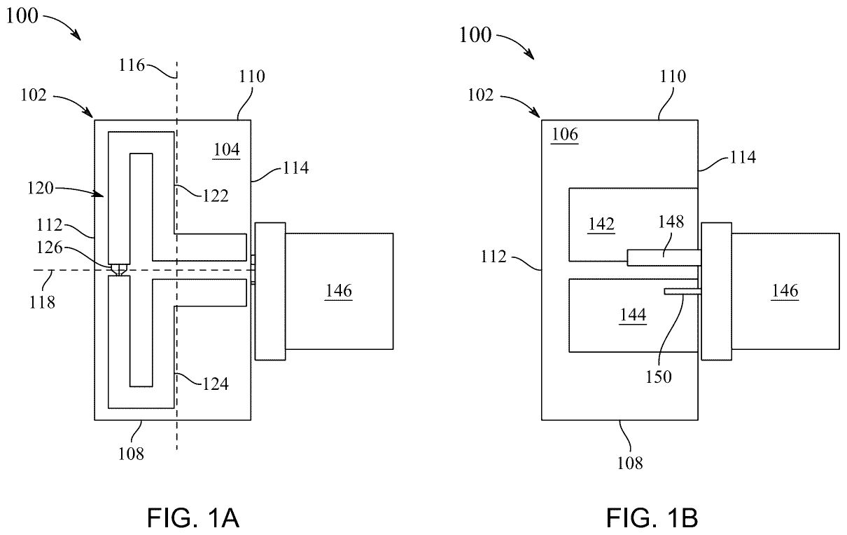

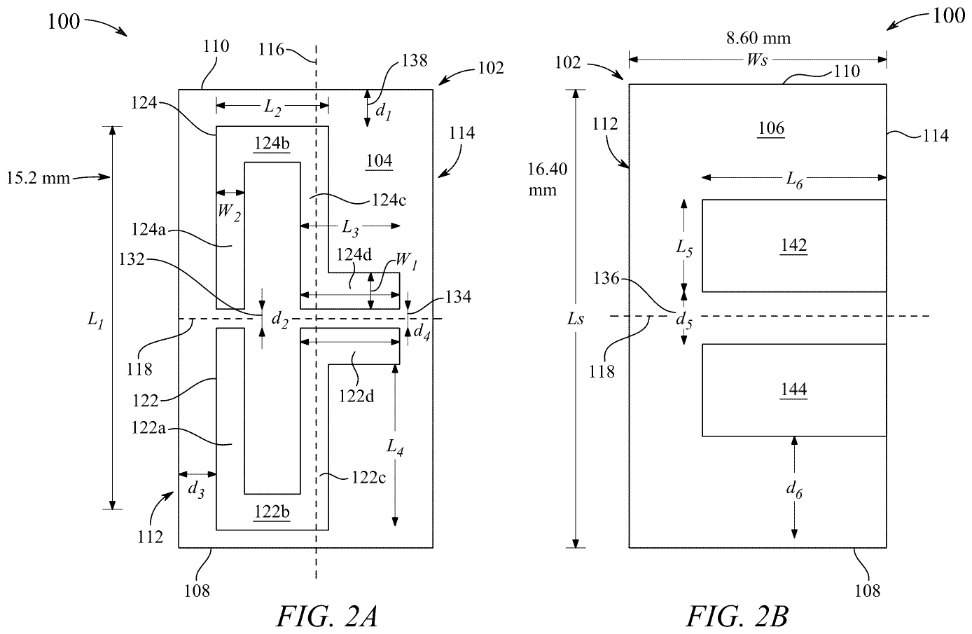

FIG. 1A-FIG. 1B illustrate an overall configuration of a folded dipole patch antenna 100. FIG. 1A may be read in conjunction with FIG. 2A-FIG. 2B for a better understanding. In the drawings of FIG. 1A-FIG. 2B, the dimensions shown are for the example of a substrate (dielectric circuit board) having a surface area of 16.40×8.60 mm² and should not be construed as limiting. For a substrate having a surface area less than or greater than 16.40×8.60 mm², the antenna dimensions are proportionately smaller or larger respectively.

FIG. 1A is a top view (front side) of the folded dipole patch antenna 100, (hereinafter interchangeably referred to as “the antenna 100”), according to one or more aspects of the present disclosure. FIG. 1B is a bottom view (back side) of the antenna 100, according to certain embodiments.

As shown in FIG. 1A and FIG. 1B, the antenna 100 includes a dielectric circuit board 102, a folded dipole microstrip antenna 120, a lumped inductor 126, a pair of parallel metallic strips (142, 144), and a coaxial feed port 146.

The dielectric circuit board 102 has a top side 104, a bottom side 106, a first edge 108, a second edge 110, a third edge 112, and a fourth edge 114. The first edge 108 is parallel to the second edge 110. The third edge 112 is perpendicular to the first edge 108 and the second edge 110. The third edge 112 is parallel to the fourth edge 114. A first central axis 116 extends from the first edge 108 to the second edge 110, and a second central axis 118 extends from the third edge 112 to the fourth edge 114. In an example, the dielectric circuit board 102 is a flame retardant (FR)-4 lossy dielectric plate. FR-4 (or FR4) is a glass-reinforced epoxy laminate material. FR-4 is a composite material composed of woven fiberglass cloth with an epoxy resin binder that is flame resistant (self-extinguishing). In an example, a thin layer of copper foil is typically laminated to one or both sides of the FR-4 lossy dielectric plate. In an example, the dielectric circuit board 102 has a length of about 16.40 mm from the first edge 108 to the second edge 110 along the first central axis 116. In an example, the dielectric circuit board 102 has a width of about 8.60 mm from the third edge 112 to the fourth edge 114 along the second central axis 118. In an example, the dielectric circuit board 102 has dimensions of about 1.52 mm in a depth direction from the top side 104 to the bottom side 106.

The folded dipole microstrip antenna 120 is formed on the top side 104. The folded dipole microstrip antenna 120 includes two meander paths (a first meander path 122, and a second meander path 124). Each of the meander paths (122, 124) has mirror geometry about the second central axis 118. As shown in FIG. 2A, a first gap 132 is centered on the second central axis 118 between the two meander paths (122,124) and near the third edge 112. A second gap 134 is centered on the second central axis 118 between the two meander paths (122, 124) near the fourth edge 114. In an example, the first gap 132 is about 0.6 mm. In an example, the second gap 134 is about 1.0 mm.

Referring to FIG. 2A, in a structural aspect, the first meander path 122 includes a first leg 122a, a second leg 122b, a third leg 122c, and an arm 122d. The first leg 122a is parallel to the third edge 112. The first leg 122a is configured to extend from the first gap 132 towards the first edge 108. In an example, the first leg 122a of the first meander path 122 is about 7.1 mm in length. The first leg 122a is parallel to and spaced from the third edge 112 by a gap d3. The second leg 122b is connected to the first leg 122a and is parallel to the first edge 108. In an example, the second leg 122b of the first meander path 122 is about 3.6 mm in length. The second leg 122b is parallel to and spaced from the first edge 108 by a distance equal to di. The third leg 122c is connected to the second leg 122b and is parallel to the first leg 122a. The third leg 122c is configured to extend to the second gap 134. In an example, the third leg 122c of the first meander path 122 is about 7.1 mm in length. The arm 122d is connected to and perpendicular to the third leg 122c. The arm 122d is configured to extend from the third leg 122c toward the fourth edge 114. In an example, the arm 122d of the first meander path 122 is about 5.15 mm in length.

As shown in FIG. 2A, in a structural aspect, the second meander path 124 is a mirror image of the first meander path about the second central axis 118. The second meander path 124 includes a first leg 124a, a second leg 124b, a third leg 124c, and an arm 124d. The first leg 124a is parallel to the third edge 112. The first leg 124a is configured to extend from the first gap 132 towards the second edge 110. In an example, the first leg 124a of the second meander path 124 is about 7.1 mm in length. The second leg 124b is connected to the first leg 124a and is parallel to the second edge 110. The second leg 124b is spaced from the second edge 110 by a fourth gap 138 which is at a distance di from the second edge 110. In an example, the second leg 124b of the second meander path 124 is about 3.6 mm in length. The third leg 124c is connected to the second leg 124b and is parallel to the first leg 124a. The third leg 124c is configured to extend to the second gap 134. In an example, the third leg 124c of the second meander path 124 is about 7.1 mm in length. The arm 124d is connected to and perpendicular to the third leg 124c. The arm 124d is configured to extend from the third leg 124c toward the fourth edge 114. In an example, the arm 124d of the second meander path 124 is about 5.15 mm in length.

As shown in FIG. 1A, the lumped inductor 126 is inserted across the first gap 132 near the third edge 112. In an example, the lumped inductor 126 has an inductance of about 200 nH.

The pair of parallel metallic strips (142, 144) is located on the bottom side 106 of the dielectric circuit board 102. The pair of parallel metallic strips (142, 144) extends from the fourth edge 114 towards the third edge 112. The pair of parallel metallic strips (142, 144) has mirror geometry about the second central axis 118. In an example, each parallel strip has a length of about 7.0 mm. Each parallel strip has a width of about 4.4 mm. A third gap 136 is located between the pair of parallel metallic strips (142, 144). In an example, the third gap 136 is about 1.0 mm.

The coaxial feed port 146 is connected to the pair of parallel metallic strips (142, 144) at the fourth edge 114. The antenna 100 is configured to resonate in a frequency of about 434 MHz upon application of an input signal at the coaxial feed port 146. A resonance frequency of 434 MHz has been found to be effective for hyperthermia applications, however, the resonance frequency can be designed by appropriate selection of the lumped inductance to be in the range of 200 MHz to 1000 Mz. The resonance frequency of the antenna may be selected to apply heat to a particular cross-section and depth of a target area, for example, a tumor within the body of a patient.

In an aspect, as shown in FIG. 1B, the antenna 100 has a first terminal end 148 and a second terminal end 150. The first terminal end 148 is connected to the coaxial feed port 146. The first terminal end 148 of the coaxial feed port 146 is connected to a first parallel strip 142 of the pair of parallel metallic strips (142, 144). The second terminal end 150 is connected to the coaxial feed port 146. The second terminal end 150 of the coaxial feed port 146 is connected to a second parallel strip 144 of the pair of parallel metallic strips (142, 144).

The defined parameters (size, operating frequency) of the folded dipole patch antenna 100 were achieved by experimenting with varying size of the components (for example, length of the meander paths (122, 124) and inductance value of the inductor 126). In an example, the antenna 100 is configured to have various defined antenna parameters, and their values are listed in table 1.

TABLE 1
Size parameters of the antenna 100

| Parameter | Value (mm) | Parameter | Value (mm) |
|---|---|---|---|
| Ls | 16.40 | Ws | 8.60 |
| L₁ | 15.2 | d₁ | 0.60 |
| L₂ | 3.60 | d₂ | 0.60 |
| L₃ | 5.15 | d₃ | 0.75 |
| L₄ | 5.60 | d₄ | 1.0 |
| L₅ | 4.40 | d₅ | 1.0 |
| L₆ | 7.0 | d₆ | 3.3 |
| W₁ | 1.50 | hs | 1.52 |

The following experiments were conducted on the antenna 100:

During experimentation, the antenna 100 was designed on a HFSS (High Frequency Structure Simulator). For example, an Ansys HFSS is a 3D electromagnetic simulation software solution for designing and simulating high-frequency electronic products such as antennas, RF and microwave components, high-speed interconnects, filters, connectors, IC components and packages and printed circuit boards. Ansys HFSS is available from RandSim, Owings Mill, Maryland, United States of America. Various experiments were performed so that a miniaturized antenna could be achieved.

During experimentation, a parametric sweep was applied to several antenna parameters (size of the folded dipole microstrip antenna 120 and pair of parallel metallic strips (142, 144), addition and deletion of multiple lumped elements) to achieve the defined antenna parameters for employing the antenna 100 in a biomedical application, such as a hyperthermia treatment for cancer. The parametric sweep was applied to observe how the results change when these parameters of the antenna change, and a parameterized design was achieved. The parametric sweep of the antenna 100 was performed to reveal how the dimensions of the components must be defined to achieve better performance.

Several antenna parameters including the length of folded dipole arm (L₃) (the arm 122d of the first meander path 122 and the arm 124d of the second meander path 124), the width (L₅) of the parallel metallic strips (142, 144) (also known as back strip width), respectively along with the inductance (L_{ind}) were varied and their corresponding reflection coefficient curves were obtained. These reflection coefficient curves were analyzed to achieve insight into the dimensions of the antenna 100.

First Experiment: Reflection Coefficient Curve of the Antenna 100 when the Input Signal is Applied to the Folded Dipole Microstrip Antenna 120.

Scattering parameters or S-parameters describe the input-output relationship between ports (or terminals) in an antenna system. For instance, in a two port antenna system, S₁₂ represents the power transferred from port 2 to port 1. S₂₁ represents the power transferred from port 1 to port 2. S₁₁ is the reflected power port 1 delivers to antenna 1 and S₂₂ is the reflected power port 2 delivers to antenna 2. Sn is known as the reflection coefficient or return loss. If S₁₁=0 dB, then all the power is reflected from the antenna 1 and nothing is radiated. If S₁₁=−10 dB, then 3 dB of power is delivered to the antenna and −7 dB is the reflected power. The remainder of the power was “accepted by” or delivered to the antenna. This accepted power is either radiated or absorbed as losses within the antenna. Since antennas are typically designed to be low loss, ideally the majority of the power delivered to the antenna is radiated. The antenna bandwidth is defined as the frequency range where Sn is less than −6 dB.

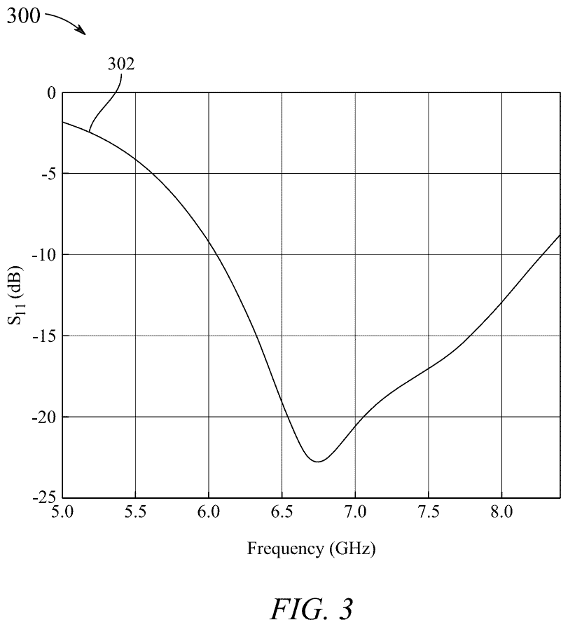

During the first experiment (a first step towards miniaturization of the antenna), initially, the antenna 100 was analyzed without adding any reactive component (inductor and capacitor). FIG. 3 is a graph 300 of the reflection coefficient curve having s-parameters (S₁₁) when the antenna 100 is without a reactive component and is fed at the top side 104 of the dielectric circuit board 102. Initially, the input signal (feed) is applied to the folded dipole microstrip antenna 120 via the coaxial feed port 146, and no reactive component is added to the antenna 100. During the first experiment, it was determined that the position of the feed port was important. Signal 302 represents the simulated values of the S₁₁ of the antenna 100. The antenna 100 resonated at the 6.75 GHz band as shown in FIG. 3.

Second Experiment: Reflection Coefficient Curve of the Antenna 100 when the Input Signal is Applied to the Pair of Parallel Metallic Strips (142, 144)

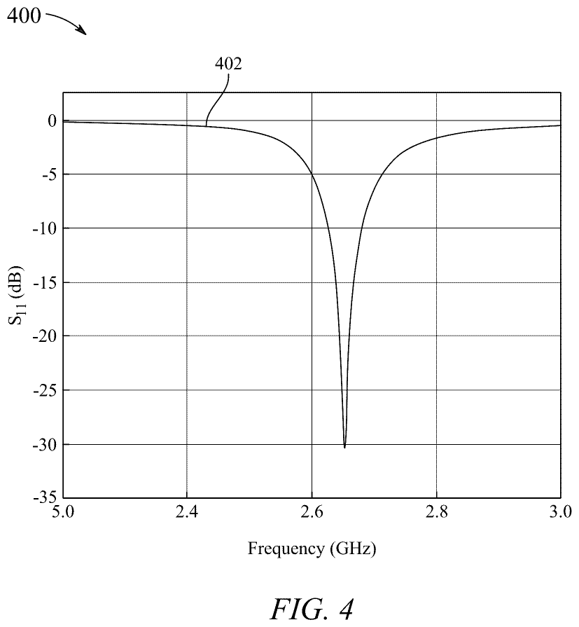

During the second experiment (a second step towards miniaturization of the antenna), instead of at the folded dipole patch arms, the antenna 100 was fed at the pair of parallel metallic strips (142, 144) (bottom side of dielectric circuit board 102). FIG. 4 is a graph 400 of the S₁₁ reflection coefficient curve when the antenna 100 without a reactive component is fed at the bottom side 106. Signal 402 represents the simulated values of the S₁₁ of the antenna 100. The change of the feeding point results in a frequency shift towards 2.65 GHz along which indicates improved impedance matching, as illustrated in FIG. 4.

### Third Experiment: Reflection Coefficient Curve of the Antenna Having Multiple Lumped Elements.

During the third experiment (a third step towards miniaturization of the antenna 100), multiple lumped elements were added to the antenna and corresponding reflection coefficient curves of s-parameters (S₁₁) were analyzed. The addition of the multiple lumped elements resulted in a shift of the resonance frequency of the antenna to the sub-1 GHz frequency band.

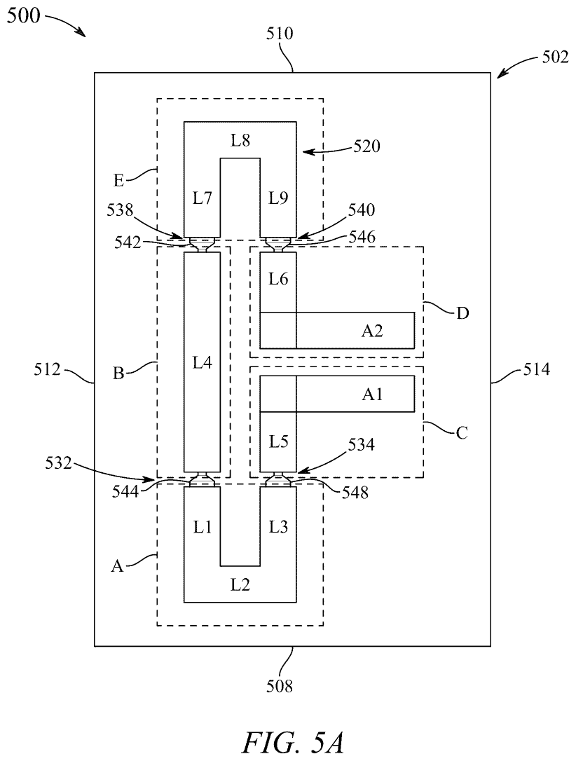

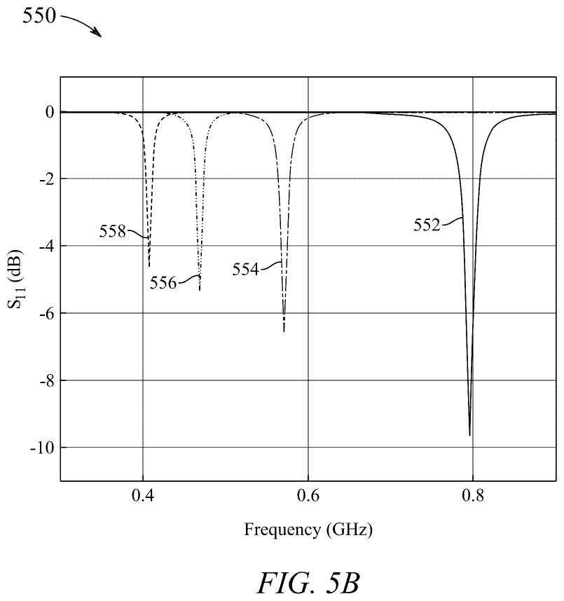

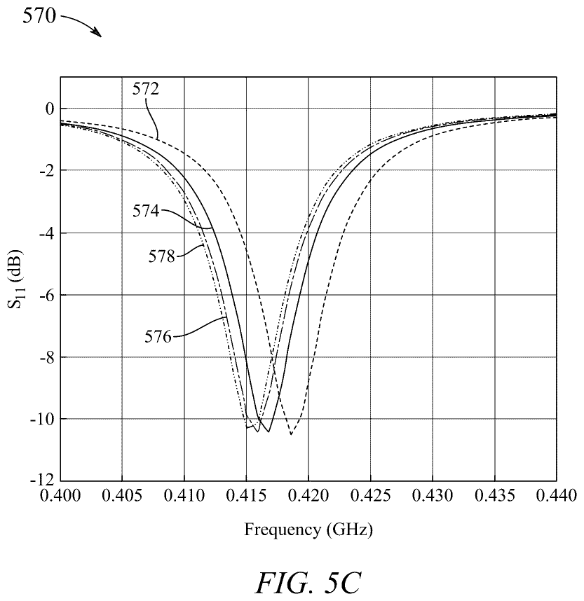

FIG. 5A is a top view of the folded dipole patch antenna 500 with multiple lumped elements. As shown in FIG. 5A, four lumped elements are added in the folded dipole microstrip antenna 520. The four lumped elements include two inductors (542, 544) and two capacitors (546, 548). The input signal is applied to the pair of parallel metallic strips located on the bottom side of the dielectric circuit board 502.

In a structural aspect, the folded dipole microstrip antenna 520 includes five sections named as a first section (A), a second section (B), a third section (C), a fourth section (D), and a fifth section (E). The first section (A) includes a first leg L₁, a second leg L₂, and a third leg L₃. The first leg L1 is parallel to the third edge 512. The second leg L2 is connected to the first leg L1 and is parallel to the first edge 508. The third leg L3 is connected to the second leg L2 and parallel to the first leg L1.

The second section (B) includes a straight leg L4. The straight leg L4 is parallel to the third edge 512. The second section (B) has a first end separated from a first end of the first leg L1 of the first section (A) by a first gap 532. A first inductor 544 is located in the first gap 532. In an example, the first inductor 544 has an inductance selected from the range of 0.4 nH to 0.8 nH.

The third section (C) includes a leg L5, and an arm A1. The leg L5 is parallel to the fourth edge 514. The arm A1 is perpendicular to and connected to the leg L5. The arm A1 is configured to extend towards the fourth edge 514. A first end of the leg L5 is separated by a second gap 534 from a second end of the first section (A). A first capacitor 548 is located in the second gap 534. In an example, the first capacitor 548 has a capacitance selected from the range of 20 pF to 80 pF.

The fourth section (D) includes a leg L6, and an arm A2. The leg L6 is parallel to the fourth edge 514. The arm A2 is perpendicular to and connected to the leg L6. The arm A2 is configured to extend towards the fourth edge 514. The arm A2 of the fourth section (D) is separated from the arm A1 of the third section (C) by a third gap 536.

The fifth section (E) includes a first leg L7, a second leg L8, and a third leg L9. The first leg L7 is parallel to the third edge 512. The second leg L8 is connected to the first leg L7 and parallel to the second edge 510. The third leg L9 is connected to the second leg L8 and parallel to the first leg L7. A first end of the fifth section (E) is separated by a fourth gap 538 from the straight leg of the second section (B). A second inductor 542 is located in the fourth gap 538. In an example, the second inductor 542 has an inductance selected from the range of 0.4 nH to 0.8 nH. A second end of the fifth section (E) is separated by a fifth gap 540 from a first end of the leg of the fourth section (D). A second capacitor 546 is located in the fifth gap 540. In an example, the second capacitor 546 has a capacitance selected from the range of 20 pF to 80 pF.

In an aspect, inductance values of the first inductor 544 and the second inductor 544 and capacitance values of the first capacitor 548 and the second capacitor 546 are selected such that the antenna 500 resonates at a frequency of about 0.434 GHz upon application of an input signal at the coaxial feed port.

FIG. 5B-FIG. 5C represent reflection coefficient curves of the antenna 500 having multiple lumped elements with varying values. A parametric sweep was applied to the inductor/capacitor and the effect on the S₁₁ parameter is illustrated in FIG. 5B-FIG. 5C. FIG. 5B is a graph 550 of the reflection coefficient curves having s-parameters (S₁₁) when the antenna 500 with multiple lumped elements has fixed capacitance and variable inductance. Signal 552 represents the simulated values of the S₁₁ when the inductance (Lind) is 40 nH. Signal 554 represents the simulated values of the S₁₁ when the inductance (Lind) is 80 nH. Further, signal 556 represents the simulated values of the S₁₁ when the inductance (Lind) is 120 nH. Signal 558 represents the simulated values of the S₁₁ when the inductance (Lind) is 160 nH. Therefore, it is clear that a larger inductance value is needed to bring the resonance frequency towards the desired frequency of 434 MHz.

FIG. 5C is a graph 570 of the reflection coefficient curves having s-parameters (S₁₁) when the antenna 500 with multiple lumped elements has fixed inductance and variable capacitance. Signal 572 represents the simulated values of the S₁₁ when the capacitance (Cₐₚ) is 40 pF. Signal 574 represents the simulated values of the Sn when the capacitance (Cₐₚ) is 80 pF. Further, signal 576 represents the simulated values of the S₁₁ when the capacitance (Cₐₚ) is 120 pF. Signal 578 represents the simulated values of the S₁₁ when the capacitance (Cₐₚ) is 160 pF. Therefore, it is clear that higher capacitance values reduce the resonance frequency, however all capacitance values reduced the resonance frequency below the target frequency of 434 MHz.

FIG. 5B shows that a higher value of inductance shifts the resonance below the required frequency, while a small value of capacitance brings the resonance curve towards the required frequency, as shown in FIG. 5C. However, the use of multiple lumped elements adds complexity to the antenna design, and balancing the values is difficult by experiments using parametric analysis. Therefore, to reduce the complexity of the multiple lumped elements, the four lumped elements are replaced with a single lumped inductor 126, as shown in FIG. 1A. The selection of the value of the lumped inductor 126 through parametric analysis resulted in a frequency shift from 2.65 GHz to 0.43 GHz.

### Fourth Experiment: Reflection Coefficient Curve of the Antenna 100 Having Single Lumped Element.

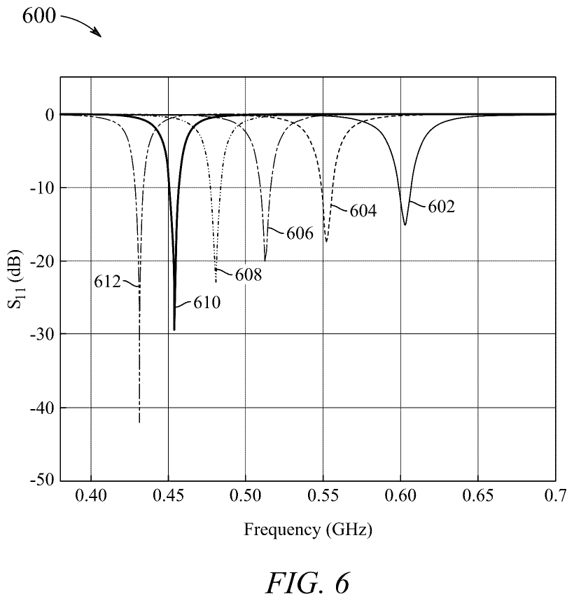

During the fourth experiment, the inductance of the antenna 100 was varied by changing the inductance value of the lumped inductor 126 shown in FIG. 1A. FIG. 6 is a graph 600 of the simulated reflection coefficient curves having s-parameters (S₁₁) for variations in the inductance (L_{ind}) of the lumped inductor 126. Signal 602 represents the simulated values of the S₁₁ when the inductance (L_{ind}) is 100 nH. Signal 604 represents the simulated values of the S₁₁ when the inductance (L_{ind}) is 120 nH. Further, signal 606 represents the simulated values of the S₁₁ when the inductance (L_{ind}) is 140 nH. Signal 608 represents the simulated values of the S₁₁ when the inductance (L_{ind}) is 160 nH. Further, signal 610 represents the simulated values of the S₁₁ when the inductance (L_{ind}) is 180 nH. Signal 612 represents the simulated values of the S₁₁ when the inductance (L_{ind}) is 200 nH.

The single inductor lumped element (lumped inductor 126) was introduced in the folded dipole microstrip antenna 120 in combination with the pair of parallel metallic strips (142, 144) and is used to shift the operating frequency to the sub-1 GHz band. The position of the lumped inductor 126 on the folded dipole microstrip antenna 120 does not create any difference in the shifting of the resonance frequency. As shown in FIG. 6, variations in inductor value were between 100 nH and 200 nH. Applying the parametric analysis to the inductor parameter shows that increasing the value of the inductor decreases the resonance frequency. Hence, when the resonance frequency is at 2.5 GHz and a 200 nH inductor is added to the antenna 100, the resonance shifted from 2.4 GHz to the sub-1 GHz band (about 434 MHz), which covers the ISM band suitable for hyperthermia applications.

### Fifth Experiment: Variations in the Length (L₃) of the First Arm 122d of the First Meander Path 122.

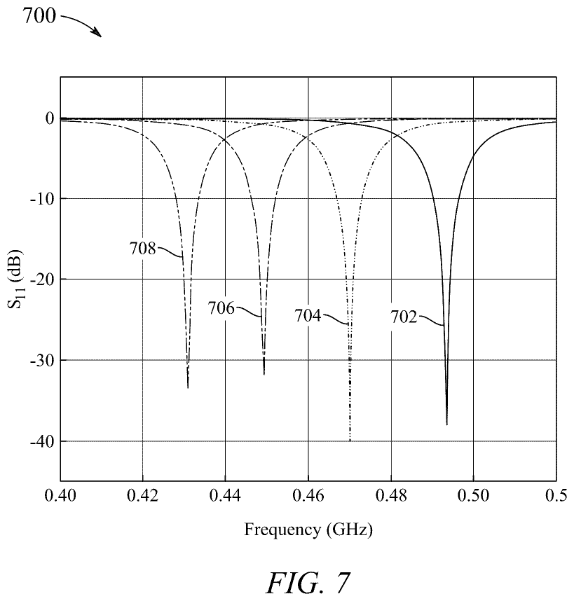

During the fifth experiment, the length (L₃) of the first arm 122d of the first meander path 122 was varied. FIG. 7 is a graph 700 representing an effect of variations in the arm length (L₃) on the reflection coefficient curves. Signal 702 represents the simulated values of the S₁₁ when the length (L₃) is 1 mm. Signal 704 represents the simulated values of Su when the length (L₃) is 2 mm. Signal 706 represents the simulated values of the S₁₁ when the length (L₃) is 3 mm. Signal 708 represents the simulated values of Su when the length (L₃) is 4 mm. From FIG. 7, it is evident that a change in the length (L₃) of the first arm 122d causes an increase or decrease in the electrical length of the folded dipole microstrip antenna 120, resulting in frequency shifting. A reduction in length shifted the resonance to higher frequencies, while an increase in arm length shifted the resonance at low frequencies.

### Sixth Experiment: Variations in the Width (L₅) of the Parallel Metallic Strips 142, 144

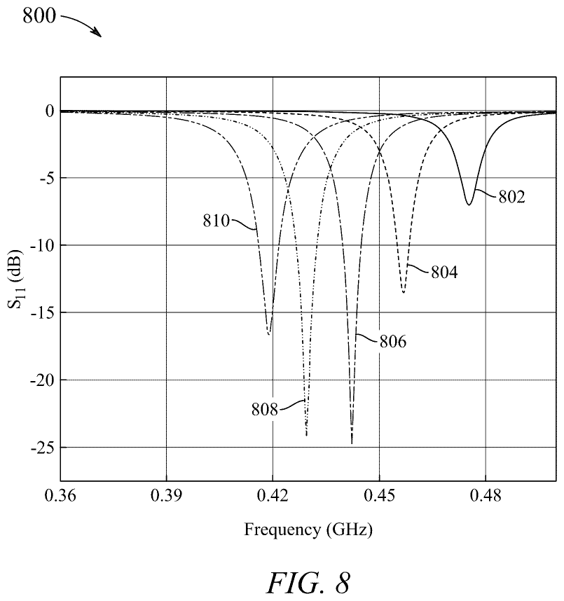

During the sixth experiment, the width (L₅) of the parallel metallic strips 142, 144 was varied. FIG. 8 is a graph 800 representing the effect of variations in width (L₅) on the reflection coefficient curves. Signal 802 represents the simulated values of the S₁₁ when the width (L₅) is 1.5 mm. Signal 804 represents the simulated values of S₁₁ when the width (L₅) is 2.5 mm. Signal 806 represents the simulated values of the S₁₁ when the width (L₅) is 3.5 mm. Signal 808 represents the simulated values of Sn when the width (L₅) is 4.5 mm. Signal 810 represents the simulated values of S₁₁ when the width (L₅) is 5.5 mm. As determined from FIG. 8, the variations in the width (L₅) of the parallel metallic strips 142, 144 have an almost similar effect to that of the change in length of the parallel metallic strips 142, 144. An improved impedance matching was achieved when the width (L₅) of the parallel metallic strips 142, 144 lay in a range of 3.5 mm to 4.5 mm, as shown in FIG. 8.

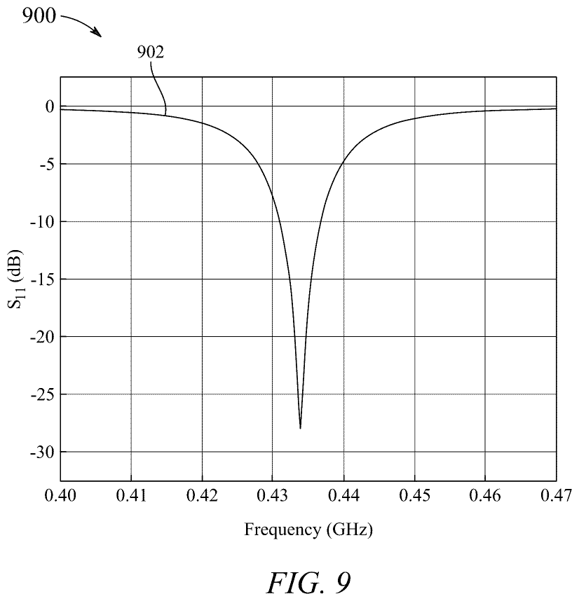

FIG. 9 is a graph 900 of a simulated reflection coefficient curve having s-parameters (S₁₁) of the antenna 100. Signal 902 represents the simulated values of S₁₁. As shown in FIG. 9, the resonance of the antenna 100 was shifted from 2.65 GHz to sub-1 GHz band (434 MHz). A defined value of the lumped inductor 126 was chosen after various parametric sweeps. As shown in FIG. 9, a bandwidth of 6 MHz is achieved using the antenna 100.

For hyperthermia applications, an antenna analysis requires the antenna performance in terms of specific absorption rate (SAR), an electrical field measurement that measures the penetration depth (PD), and in terms of effective field strength (EFS). For EFS and SAR measurements, the HFSS tool is used, where the antenna 100 is placed near a hand region and the required performance is measured by measuring the input power as 1 W for 1 g of tissue. The units for SAR measurements are watts per kilogram (W/kg). The units for EFS measurements are volts per meter (V/m). The variations in a distance between the antenna 100 and the hand resulted in altered SAR and EFS values. As the distance increased, the SAR and PD decreased while EFS increased. Also, a shift in resonance frequency also depends on the distance of the antenna from the body.

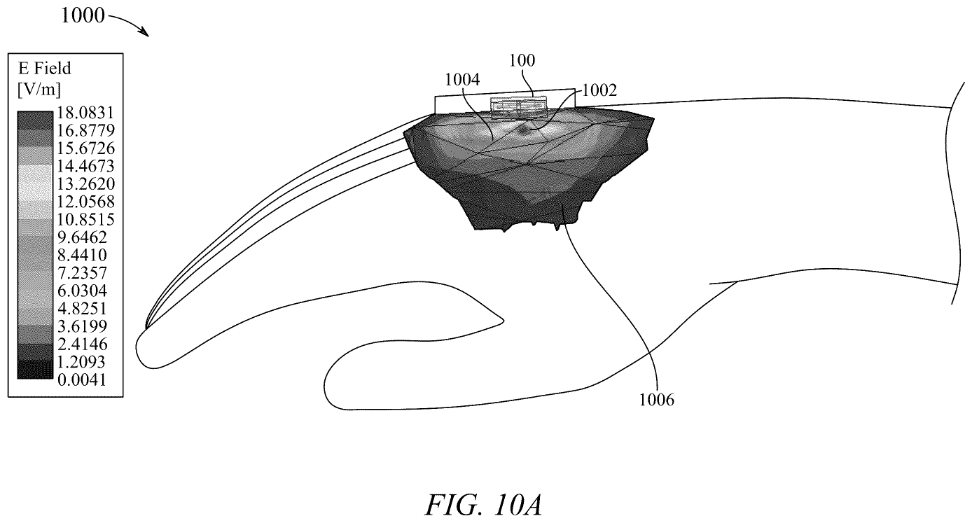

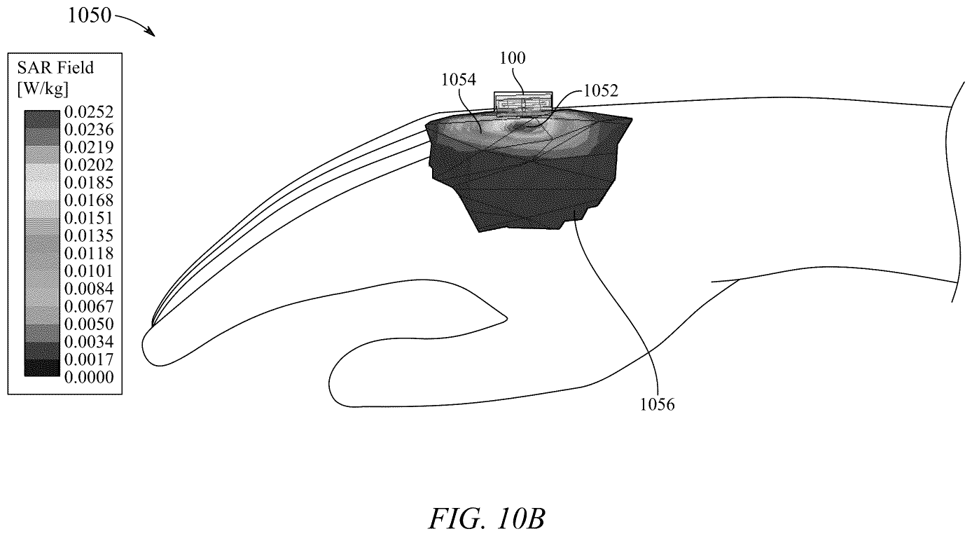

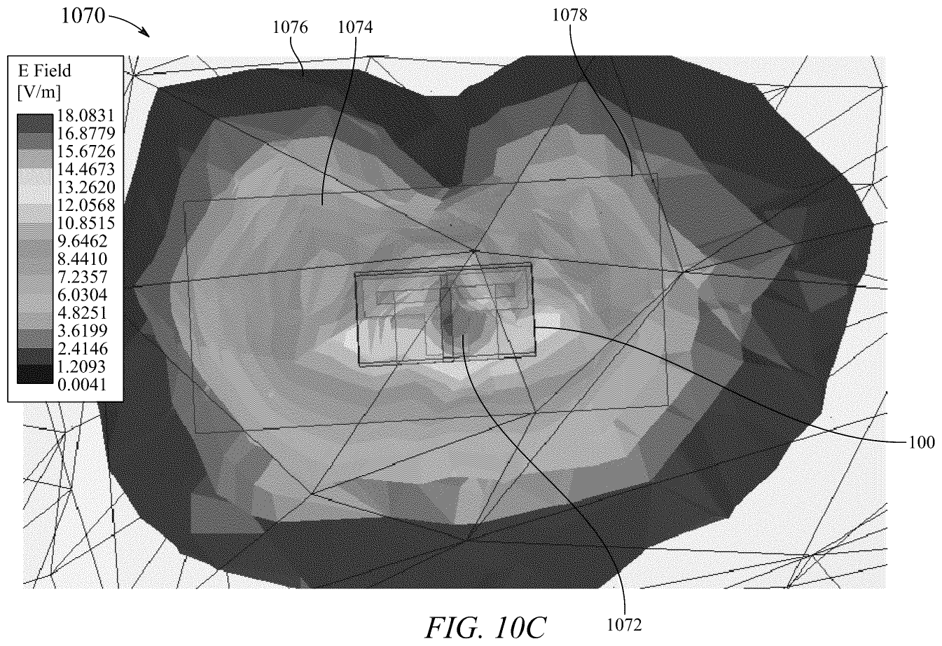

FIG. 10A-FIG. 10C demonstrate the simulated results of the electric field and SAR field on a human hand. When the antenna 100 is placed on a body part, the most important parameter is the SAR measurement which indicates the radiation effect on the human body. The SAR measuring formula includes electric field information to measure the SAR. FIG. 10A and FIG. 10B represent the electric field and SAR measurements inside the human hand, while FIG. 10C represents the EFS of the antenna 100.

FIG. 10A is a representation 1000 of the measured electric field distribution when the antenna 100 is placed on the human hand. As shown in FIG. 10A, the measured electric field distribution can be divided into three main areas i.e., a high intensity area 1002, a medium intensity area 1004, and a low intensity area 1006. For example, the high intensity area 1002 had an electric field of 18.08 V/m to 15.67 V/m, the medium intensity area 1004 had an electric field of 12.05 V/m to 7.23 V/m, and the low intensity area 1006 had an electric field of 2.4 V/m to 0.004 V/m.

FIG. 10B is a representation 1050 of measured SAR field when the antenna 100 is placed on the human hand. As shown in FIG. 10B, the measured SAR field can be divided into three main areas i.e., a high intensity area 1052, a medium intensity area 1054, and a low intensity area 1056. For example, the high intensity area 1052 had a SAR field of 0.0252 W/kg to 0.0236 W/kg, the medium intensity area 1054 has a SAR field of 0.0168 W/kg to 0.0101 W/kg, and the low intensity area 1056 has a SAR field of 0.0017 W/kg to 0.00 W/kg.

FIG. 10C is a top view 1070 of the EFS when the antenna 100 is placed on the human hand. As shown in FIG. 10C, the measured EFS can be divided into three main areas i.e., a high intensity area 1072, a medium intensity area 1074, and a low intensity area 1076. For measurement of EFS, a rectangular section 1078 is considered around the electric field at around 1 cm depth. In an example, the measured area of the rectangular section 1078 is 50 mm×26 mm. The rectangular section 1078 is configured to cover the high intensity area 1072, and the medium intensity area 1074. For example, the high intensity area 1072 had an electric field of 18.08 V/m to 15.67 V/m, the medium intensity area 1074 had an electric field of 12.05 V/m to 7.23 V/m, and the low intensity area 1076 had an electric field of 2.4 V/m to 0.004 V/m.

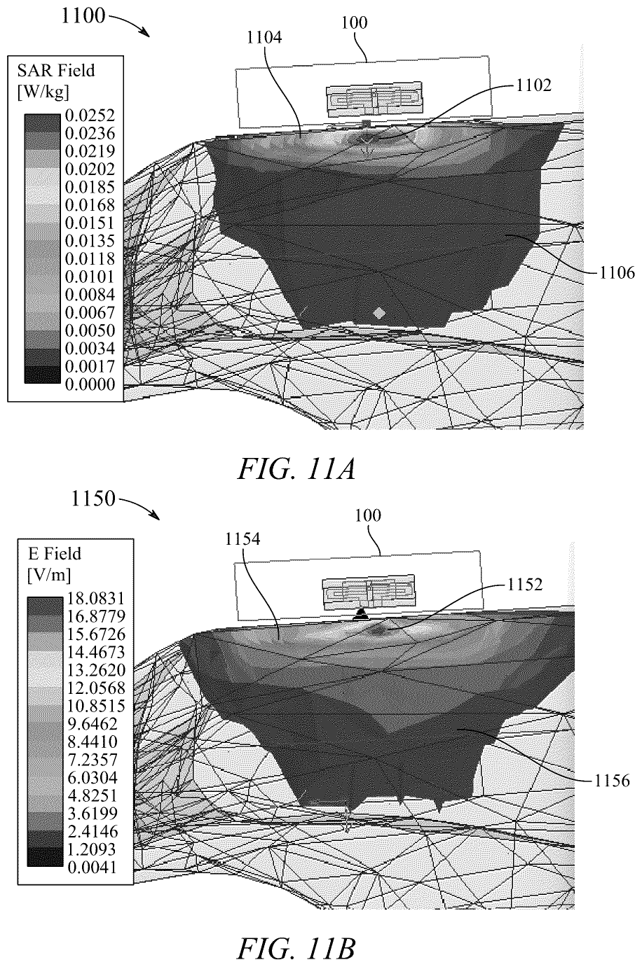

Penetration depth was measured when the antenna 100 was placed on the hand as shown in FIG. 11A-FIG. 11B. Further, the HFSS (distance tool) is used to measure the penetration depth of the antenna from the skin, which is approximately 70 mm.

FIG. 11A is a representation 1100 of the penetration depth of the antenna 100 inside the human hand calculated using the SAR field. As shown in FIG. 11A, the measured SAR field can be divided into three main areas depending upon SAR intensity i.e., a high intensity area 1102, a medium intensity area 1104, and a low intensity area 1106. For example, the high intensity area 1102 had SAR intensity in a range of 0.0252 W/kg to 0.0236 W/kg. The high intensity area 1102 had a penetration depth of 70 mm, the medium intensity area 1104 had SAR intensity in a range of 0.0168 W/kg to 0.0101 W/kg, and the low intensity area 1106 has SAR intensity in a range of 0.0017 W/kg to 0.00 W/kg.

FIG. 11B is a representation 1150 of the penetration depth of the antenna 100 inside the human hand using the electric field. As shown in FIG. 11B, the measured electric field can be divided into three main areas depending upon penetration depth i.e., a high intensity area 1152, a medium intensity area 1154, and a low intensity area 1156. For example, the high intensity area 1002 had an electric field in a range of 18.08 V/m to 15.67 V/m. The high intensity area 1152 has a penetration depth of 71.17 mm. In an example, the medium intensity area 1004 had an electric field in a range of 12.05 V/m to 7.23 V/m. In an example, the low intensity area 1006 had an electric field in a range of 2.4 V/m to 0.004 V/m.

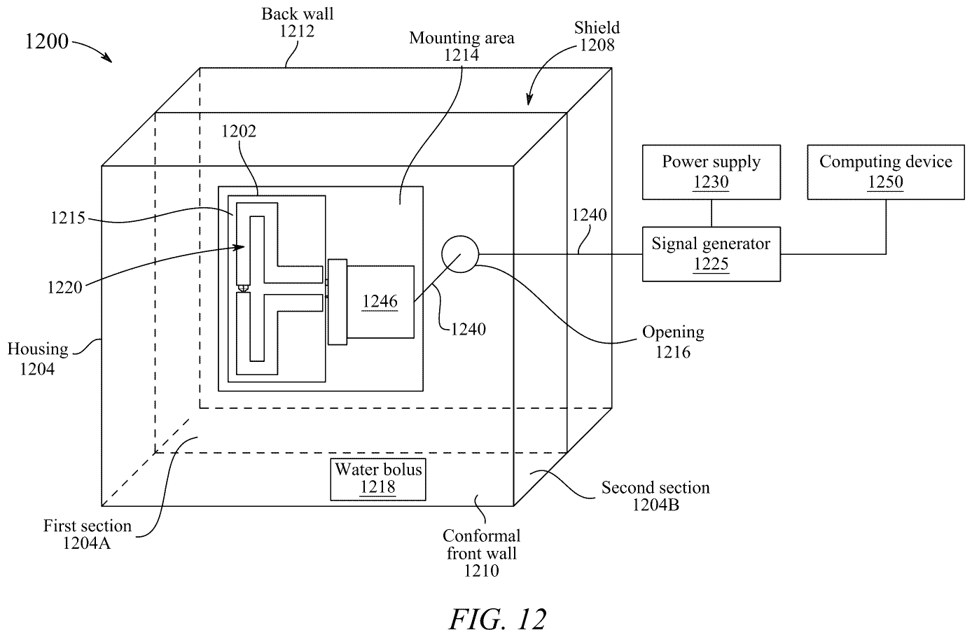

FIG. 12 represents a block diagram of a hyperthermia applicator 1200 for use in hyperthermia medical treatments. The hyperthermia applicator 1200 is configured to emit microwave energy toward a target tissue (cancer tumor). In an example, the hyperthermia applicator 1200 includes a signal generator 1225, a hyperthermia antenna 1215, a power supply 1230, and a coaxial cable 1240. In an aspect, the hyperthermia applicator 1200 is connected to a remotely placed computing device 1250. The computing device 1250 may be any device, such as a desktop computer, a laptop, a tablet computer, a smartphone, an imaging device, a smart watch, a mobile device, a Personal Digital Assistant (PDA) or any other computing device. The computing device 1250 is configured to provide a real-time feedback as to the position of the hyperthermia applicator 1200 relative to the target tissue and thermometry data so as to permit real-time adjustment of the operating parameters of the hyperthermia applicator 1200.

The computing device 1250 is also connected to at least one thermal sensor (not shown in FIG. 12) placed in proximity of the target tissue. The at least one thermal sensor is configured to measure the temperature of the target tissue to be heated. To conduct the treatment, the temperature inside the tumor must be monitored. To monitor the internal temperature, a thermal sensor (for example, a micro thermometer) is inserted through the skin into the tumor.

In operative aspects, the hyperthermia antenna 1215 may be an invasive antenna or a non-invasive antenna. The hyperthermia applicator 1200 (hyperthermia antenna 1215) includes a dielectric circuit board 1202, a folded dipole microstrip antenna 1220, a first gap, a lumped inductor, a second gap, a pair of parallel metallic strips, a third gap, and a coaxial feed port 1246. The construction of the various components (such as the dielectric circuit board 1202, the folded dipole microstrip antenna 1220, the first gap, the lumped inductor, the second gap, the pair of parallel metallic strips, the third gap) of the hyperthermia applicator 1200 (hyperthermia antenna 1215), as shown in FIG. 12, is substantially similar to the folded dipole patch antenna 100 of FIG. 1A, and thus the construction is not repeated here in detail for the sake of brevity.

In an example, the dielectric circuit board 1202 has dimensions of about 1.52 mm in a depth direction from the top side to the bottom side.

The folded dipole microstrip antenna 1220 is formed on the top side. In an example, the folded dipole microstrip antenna 1220 is fabricated from copper because of its availability, good conductivity, corrosion resistance and low-cost. In another example, the folded dipole microstrip antenna 1220 may be fabricated from gold, silver, graphene, conductive polymers and aluminum.

The lumped inductor is inserted across the first gap near the third edge. In an example, the lumped inductor has an inductance of about 200 nH.

The pair of parallel metallic strips is located on the bottom side and is connected to the coaxial feed port 1246.

The signal generator 1225 is connected to the power supply 1230. The signal generator 1225 is configured to generate an alternating voltage in a microwave frequency range.

In an operative aspect, the thermal sensor provides feedback to the computing device 1250 regarding the temperature of the target tissue. Based on the feedback, the computing device 1250 is configured to generate a signal to be transmitted to the signal generator 1225 such that the amplitude of the microwave signals may be regulated and overheating of the patient's body can be prevented.

The coaxial cable 1240 is connected to the signal generator 1225.

The coaxial feed port 1246 is connected to the coaxial cable 1240 at a receiving end. The coaxial feed port 1246 is connected to the pair of parallel metallic strips. The hyperthermia antenna 1215 (folded dipole patch antenna) is configured to resonate in a frequency range of about 434 MHz upon receiving the alternating voltage in the microwave frequency range at the coaxial feed port 1246. In an aspect, the coaxial feed port 1246 is configured with a signal conduction terminal and a ground terminal. The signal conduction terminal is connected to a first parallel strip of the pair of parallel metallic strips, and the ground terminal is connected to a second parallel strip of the pair of parallel metallic strips.

As shown in FIG. 12, the hyperthermia applicator includes a housing 1204, a hermetic, electrically transparent shield 1208, and a conformal front wall 1210. The housing 1204 includes a back wall 1212 which is configured with a mounting area 1214. The mounting area 1214 is configured to hold the dielectric circuit board 1202. The back wall 1212 also includes an opening 1216 which is configured to permit the coaxial feed port 1246 to protrude through the back wall 1212. The hermetic, electrically transparent shield 1208 is configured to separate the housing 1204 into two sections i.e., a first section 1204A and a second section 1204B. The first section 1204A includes the dielectric circuit board 1202. The second section 1204B is configured to hold a water bolus 1218. The water bolus 1218 is configured to couple the electromagnetic (EM) energy, released by the hyperthermia antenna 1215, into the target tissue and cools the surface of the tissue to minimize thermal hotspots and patient discomfort during cancer treatment. In an example, the water bolus 1218 is filled with demineralized water. The conformal front wall 1210 is configured to hermetically seal the water bolus 1218 within the second section 1204B. The water bolus 1218 is configured to prevent the hyperthermia applicator from burning the skin by preventing direct contact with the antenna.

The present disclosure describes a miniaturized folded dipole patch antenna 100 suitable for hyperthermia and biomedical applications. The antenna 100 is electrically small, without a balun transformer, and has a 502 impedance without using an impedance matching circuit. The antenna 100 resonates at 6.8 GHz, however changing the position of the feed points and adding the lumped inductive element to the folded dipole patch, lowers the resonance notch to 434 MHz. During experiments, the antenna 100 was attached to a human hand model in HFSS, where the SAR, EFS, and penetration depth were evaluated. The antenna 100 provides good penetration depth along with EFS greater than the actual antenna size. Antenna simplicity, miniature size, improved EFS, and penetration depth are the key features of the present folded dipole patch antenna.

The first embodiment is illustrated with respect to FIG. 1A-FIG. 2B. The first embodiment describes the folded dipole patch antenna 100. The folded dipole patch antenna 100 includes a dielectric circuit board 102, a folded dipole microstrip antenna, a first gap 132, a lumped inductor, a second gap 134, a pair of parallel metallic strips (142, 144), a third gap 136, and a coaxial feed port 146. The dielectric circuit board 102 includes a top side 104, a bottom side 106, a first edge 108, a second edge 110 parallel to the first edge 108, a third edge 112 perpendicular to the first edge 108 and the second edge 110, a fourth edge 114 parallel to the third edge 112, a first central axis 116 which extends from the first edge 108 to the second edge 110, and a second central axis 118 which extends from the third edge 112 to the fourth edge 114. The folded dipole microstrip antenna 120 is formed on the top side 104. The folded dipole microstrip antenna 120 includes two meander paths, each having mirror geometry about the second central axis 118. The first gap 132 is centered on the second central axis 118 between the two meander paths and near the third edge 112. The lumped inductor is inserted across the first gap 132 near the third edge 112. The second gap 134 is centered on the second central axis 118 between the two meander paths near the fourth edge 114. The pair of parallel metallic strips (142, 144) is located on the bottom side 106. The pair of parallel metallic strips (142, 144) extends from the fourth edge 114 towards the third edge 112. The pair of parallel metallic strips (142, 144) has mirror geometry about the second axis. The third gap 136 is located between the pair of parallel metallic strips (142, 144). The coaxial feed port 146 is connected to the pair of parallel metallic strips (142, 144) at the fourth edge 114. The folded dipole patch antenna is configured to resonate in a frequency range of about 434 MHz upon application of an input signal at the coaxial feed port 146.

In an aspect, a length of the dielectric circuit board 102 between the first edge 108 and second edge 110 is about 16.4 mm and a width of the dielectric circuit board 102 between the third edge 112 and the fourth edge 114 is about 8.6 mm, and the lumped inductor has an inductance of about 200 nH.

In an aspect, a first meander path 122 of the two meander paths includes a first leg parallel to the third edge 112, a second leg connected to the first leg and parallel to the first edge 108, a third leg connected to the second leg and parallel to the first leg, and an arm connected to and perpendicular to the third leg. The first leg is configured to extend from the first gap 132 towards the first edge 108. The second leg is spaced from the first edge 108 by a third gap 136. The third leg is configured to extend to the second gap 134. The arm is configured to extend from the third leg toward the fourth edge 114.

In an aspect, a second meander path 124 of the two meander paths includes a first leg parallel to the third edge 112, a second leg connected to the first leg and parallel to the second edge 110, a third leg connected to the second leg and parallel to the first leg, and an arm connected to and perpendicular to the third leg. The first leg is configured to extend from the first gap 132 towards the second edge 110. The second leg is spaced from the second edge 110 by a fourth gap 138. The third leg is configured to extend to the second gap 134. The arm is configured to extend from the third leg toward the fourth edge 114.

In an aspect, the first leg of the first meander path 122 is about 7.1 mm in length, the second leg of the first meander path 122 is about 3.6 mm in length, the third leg of the first meander path 122 is about 7.1 mm in length, and the arm of the first meander path 122 is about 5.15 mm in length.

In an aspect, the first leg of the second meander path 124 is about 7.1 mm in length, the second leg of the second meander path 124 is about 3.6 mm in length, the third leg of the second meander path 124 is about 7.1 mm in length, and the arm of the second meander path 124 is about 5.15 mm in length.

In an aspect, the first gap 132 is about 0.6 mm, and the second gap 134 is about 1.0 mm.

In an aspect, each parallel strip has a length of about 7.0 mm, each parallel strip has a width of about 4.4 mm, and the third gap 136 is about 1.0 mm.

In an aspect, the antenna 100 includes a first terminal end connected to the coaxial feed port 146, and a second terminal end connected to the coaxial feed port 146. The first terminal end of the coaxial feed port 146 is connected to a first parallel strip of the pair of parallel metallic strips (142, 144). The second terminal end of the coaxial feed port 146 is connected to a second parallel strip of the pair of parallel metallic strips (142, 144).

The second embodiment is illustrated with respect to FIG. 12. The second embodiment describes the hyperthermia applicator for use in hyperthermia medical treatments. The hyperthermia applicator includes a dielectric circuit board 1202, a folded dipole microstrip antenna, a first gap, a lumped inductor, a second gap, a pair of parallel metallic strips, a third gap, a power supply, a signal generator, a coaxial cable, and a coaxial feed port. The dielectric circuit board 1202 has a top side, a bottom side, a first edge, a second edge parallel to the first edge, a third edge perpendicular to the first edge and the second edge, a fourth edge parallel to the third edge, a first central axis which extends from the first edge to the second edge, and a second central axis which extends from the third edge to the fourth edge. The folded dipole microstrip antenna 1220 is formed on the top side. The folded dipole microstrip antenna 1220 includes two meander paths, each having mirror geometry about the second central axis. The first gap is centered on the second central axis between the two meander paths and near the third edge. The lumped inductor inserted across the first gap near the third edge. The second gap is centered on the second central axis between the two meander paths near the fourth edge. The pair of parallel metallic strips is located on the bottom side. The pair of parallel metallic strips extends from the fourth edge 114 towards the third edge 112. The pair of parallel metallic strips has mirror geometry about the second axis. The third gap is located between the pair of parallel metallic strips. The signal generator is connected to the power supply. The signal generator is configured to generate an alternating voltage in a microwave frequency range. The coaxial cable is connected to the signal generator. The coaxial feed port 1246 is connected to the coaxial cable at a receiving end, and is connected to the pair of parallel metallic strips at the fourth edge. The folded dipole patch antenna is configured to resonate in a frequency range of about 434 MHz upon receiving the alternating voltage in the microwave frequency range at the coaxial feed port 1246 and emit microwave energy; and wherein the microwave energy is configured to raise the temperature of the target area when the hyperthermia applicator is placed over the target area of the human body.

In an aspect, a length of the dielectric circuit board 1202 between the first edge and second edge is about 16.4 mm and a width of the dielectric circuit board 1202 between the third edge and the fourth edge is about 8.6 mm, and the lumped inductor has an inductance of about 200 nH.

In an aspect, each parallel strip has a length of about 7.0 mm, each parallel strip has a width of about 4.4 mm, and the third gap 136 is about 1.0 mm.

In an aspect, the coaxial feed port 1246 is configured with a signal conduction terminal and a ground terminal, wherein the signal conduction terminal is connected to a first parallel strip of the pair of parallel metallic strips, and wherein the ground terminal is connected to a second parallel strip of the pair of parallel metallic strips.

In an aspect, a first meander path of the two meander paths includes a first leg parallel to the third edge, a second leg connected to the first leg and parallel to the first edge, a third leg connected to the second leg and parallel to the first leg, and an arm connected to and perpendicular to the third leg. The first leg is configured to extend from the first gap towards the first edge. The second leg is spaced from the first edge by a third gap. The third leg is configured to extend to the second gap. The arm is configured to extend from the third leg toward the fourth edge.

In an aspect, a second meander path of the two meander paths includes a first leg parallel to the third edge, a second leg connected to the first leg and parallel to the second edge, a third leg connected to the second leg and parallel to the first leg, and an arm connected to and perpendicular to the third leg. The first leg is configured to extend from the first gap towards the second edge. The second leg is spaced from the second edge by a fourth gap. The third leg is configured to extend to the second gap. The arm is configured to extend from the third leg toward the fourth edge.

In an aspect, the first leg of the first meander path is about 7.1 mm in length, the second leg of the first meander path is about 3.6 mm in length, the third leg of the first meander path is about 7.1 mm in length, and the arm of the first meander path is about 5.15 mm in length. In an aspect, the first leg of the second meander path is about 7.1 mm in length, the second leg of the second meander path is about 3.6 mm in length, the third leg of the second meander path is about 7.1 mm in length, and the arm of the second meander path is about 5.15 mm in length.

In an aspect, the hyperthermia applicator includes a housing comprising a back wall configured with a mounting area which holds the dielectric circuit board 1202, a hermetic, electrically transparent shield, and a conformal front wall. The back wall includes an opening sized to permit the coaxial feed port 1246 to protrude through the back wall. The hermetic, electrically transparent shield is configured to separate the housing into a first section including the dielectric circuit board 1202 and a second section. The second section is configured to hold a water bolus. The conformal front wall configured to hermetically seal the water bolus within the second section.

The third embodiment is illustrated with respect to FIG. 1A-FIG. 5C. The third embodiment describes the folded dipole patch antenna. The folded dipole patch antenna includes a dielectric circuit board 102, a folded dipole microstrip antenna, a pair of parallel metallic strips (142, 144), and a coaxial feed port 146. The dielectric circuit board 102 includes a top side, a bottom side 106, a first edge 108, a second edge 110 parallel to the first edge 108, a third edge 112 perpendicular to the first edge 108 and the second edge 110, a fourth edge 114 parallel to the third edge 112, a first central axis 116 which extends from the first edge 108 to the second edge 110, and a second central axis 118 which extends from the third edge 112 to the fourth edge 114. A length of the dielectric circuit board 102 between the first edge 108 and second edge 110 is about 16.4 mm and a width of the dielectric circuit board 102 between the third edge 112 and the fourth edge 114 is about 8.6 mm. The folded dipole microstrip antenna 120 is formed on the top side 104. The folded dipole microstrip antenna 520 includes five sections. A first section (A) includes a first leg parallel to the third edge 512, a second leg connected to the first leg and parallel to the first edge 508, and a third leg connected to the second leg and parallel to the first leg. A second section (B) includes a straight leg parallel to the third edge 512. The second section (B) has a first end separated from a first end of the first leg of the first section by a first gap 532. A third section (C) includes a leg parallel to the fourth edge 514 and an arm perpendicular to and connected to the leg. The arm is configured to extend towards the fourth edge 514. A first end of the leg is separated by a second gap 534 from a second end of the first section. A fourth section (D) includes a leg parallel to the fourth edge 514 and an arm perpendicular to and connected to the leg. The arm is configured to extend towards the fourth edge 514. The arm of the fourth section is separated from the arm of the third section by a third gap 536. A fifth section (E) includes a first leg parallel to the third edge 512, a second leg connected to the first leg and parallel to the second edge 110, and a third leg connected to the second leg and parallel to the first leg. A first end of the fifth section is separated by a fourth gap 538 from the straight section and a second end of the fifth section is separated by a fifth gap 540 from a first end of the leg of the fourth section. A first inductor is located in the first gap 532. A first capacitor is located in the second gap 534. A second inductor is located in the fourth gap 538. A second capacitor is located in the fifth gap 540. The pair of parallel metallic strips (142, 144) is located on the bottom side 106. The pair of parallel metallic strips (142, 144) is configured to extend from the fourth edge 114 towards the third edge 112. The pair of parallel metallic strips (142, 144) has mirror geometry about the second axis. The coaxial feed port 146 is connected to the pair of parallel metallic strips (142, 144) at the fourth edge 114. The inductance values of the first inductor and the second inductor and capacitance values of the first capacitor and the second capacitor are selected such that the folded dipole patch antenna resonates at a frequency of about 0.434 GHz upon application of an input signal at the coaxial feed port 146.

In an aspect, the first inductor has an inductance selected from the range of 0.4 nH to 0.8 nH, the first capacitor has a capacitance selected from the range of 20 pF to 80 pF, the second inductor has an inductance selected from the range of 0.4 nH to 0.8 nH, and the second capacitor has a capacitance selected from the range of 20 pF to 80 pF.

In an aspect, the first gap 132 is about 0.6 mm, the second gap 134 is about 1.0 mm, each parallel strip has a length of about 7.0 mm, each parallel strip has a width of about 4.4 mm, and the third gap 136 is about 1.0 mm.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1-9. (canceled)

10. A hyperthermia applicator for use in hyperthermia medical treatments to induce a temperature rise in a target area of a human body, comprising:
a dielectric circuit board including a top side, a bottom side, a first edge, a second edge parallel to the first edge, a third edge perpendicular to the first edge and the second edge, and a fourth edge parallel to the third edge, a first central axis which extends from the first edge to the second edge, and a second central axis which extends from the third edge to the fourth edge;
a folded dipole microstrip antenna formed on the top side, wherein the folded dipole microstrip antenna includes two meander paths, each having mirror geometry about the second central axis;
a first gap centered on the second central axis between the two meander paths and near the third edge;
a lumped inductor inserted across the first gap near the third edge;
a second gap centered on the second central axis between the two meander paths near the fourth edge;
a pair of parallel metallic strips located on the bottom side, wherein the pair of parallel metallic strips extends from the fourth edge towards the third edge, wherein the pair of parallel metallic strips has mirror geometry about the second axis;
a third gap located between the pair of parallel metallic strips;
a power supply;
a signal generator connected to the power supply, wherein the signal generator is configured to generate an alternating voltage in a microwave frequency range;
a coaxial cable connected to the signal generator;
a coaxial feed port connected to the coaxial cable at a receiving end, and connected to the pair of parallel metallic strips at the fourth edge,
wherein the folded dipole patch antenna is configured to resonate in a frequency range of about 434 MHz upon receiving the alternating voltage in the microwave frequency range at the coaxial feed port and emit microwave energy; and
wherein the microwave energy raises the temperature of the target area when the hyperthermia applicator is placed over the target area of the human body.

11. The hyperthermia applicator of claim 10, wherein:
a length of the dielectric circuit board between the first edge and second edge is about 16.4 mm,
a width of the dielectric circuit board between the third edge and the fourth edge is about 8.6 mm, and
the lumped inductor has an inductance of about 200 nH.

12. The hyperthermia applicator of claim 11, wherein each parallel strip has a length of about 7.0 mm, each parallel strip has a width of about 4.4 mm; and the third gap is about 1.0 mm.

13. The hyperthermia applicator of claim 12, wherein the coaxial feed port is configured with a signal conduction terminal and a ground terminal, wherein the signal conduction terminal is connected to a first parallel strip of the pair of parallel strips, and wherein the ground terminal is connected to a second parallel strip of the pair of parallel strips.

14. The hyperthermia applicator of claim 11, wherein a first meander path of the two meander paths comprises:
a first leg parallel to the third edge, wherein the first leg is configured to extend from the first gap towards the first edge;
a second leg connected to the first leg and parallel to the first edge, wherein the second leg is spaced from the first edge by a third gap;
a third leg connected to the second leg and parallel to the first leg, wherein the third leg is configured to extend to the second gap; and
an arm connected to and perpendicular to the third leg, wherein the arm is configured to extend from the third leg toward the fourth edge.

15. The hyperthermia applicator of claim 14, wherein a second meander path of the two meander paths comprises:
a first leg parallel to the third edge, wherein the first leg is configured to extend from the first gap towards the second edge;
a second leg connected to the first leg and parallel to the second edge, wherein the second leg is spaced from the second edge by a fourth gap;
a third leg connected to the second leg and parallel to the first leg, wherein the third leg is configured to extend to the second gap; and
an arm connected to and perpendicular to the third leg, wherein the arm is configured to extend from the third leg toward the fourth edge.

16. The hyperthermia applicator of claim 15, wherein:
the first leg of the first meander path is about 7.1 mm in length;
the second leg of the first meander path is about 3.6 mm in length;
the third leg of the first meander path is about 7.1 mm in length;
the arm of the first meander path is about 5.15 mm in length;
the first leg of the second meander path is about 7.1 mm in length;
the second leg of the second meander path is about 3.6 mm in length;
the third leg of the second meander path is about 7.1 mm in length; and
the arm of the second meander path is about 5.15 mm in length.

17. The hyperthermia applicator of claim 10, further comprising:
a housing comprising a back wall configured with a mounting area which holds the dielectric circuit board, the back wall including an opening sized to permit the coaxial feed port to protrude through the back wall;
a hermetic, electrically transparent shield configured to separate the housing into a first section including the dielectric circuit board and a second section, wherein the second section is configured to hold a water bolus; and
a conformal front wall configured to hermetically seal the water bolus within the second section.

18. A folded dipole patch antenna, comprising:
a dielectric circuit board including a top side, a bottom side, a first edge, a second edge parallel to the first edge, a third edge perpendicular to the first edge and the second edge, and a fourth edge parallel to the third edge, a first central axis which extends from the first edge to the second edge, and a second central axis which extends from the third edge to the fourth edge, wherein a length of the dielectric circuit board between the first edge and second edge is about 16.4 mm and a width of the dielectric circuit board between the third edge and the fourth edge is about 8.6 mm;
a folded dipole microstrip antenna formed on the top side, wherein the folded dipole microstrip antenna consists of five sections, wherein:
a first section includes a first leg parallel to the third edge, a second leg connected to the first leg and parallel to the first edge, and a third leg connected to the second leg and parallel to the first leg;
a second section includes a straight leg parallel to the third edge, wherein the second section has a first end separated from a first end of the first leg of the first section by a first gap;
a third section includes a leg parallel to the fourth edge and an arm perpendicular to and connected to the leg, wherein the arm is configured to extend towards the fourth edge, wherein a first end of the leg is separated by a second gap from a second end of the first section;
a fourth section includes a leg parallel to the fourth edge and an arm perpendicular to and connected to the leg, wherein the arm is configured to extend towards the fourth edge, wherein the arm of the fourth section is separated from the arm of the third section by a third gap;
a fifth section includes a first leg parallel to the third edge, a second leg connected to the first leg and parallel to the second edge, and a third leg connected to the second leg and parallel to the first leg, wherein a first end of the fifth section is separated by a fourth gap from the straight section and a second end of the fifth section is separated by a fifth gap from a first end of the leg of the fourth section;
a first inductor located in the first gap;
a first capacitor located in the second gap;
a second inductor located in the fourth gap;
a second capacitor located in the fifth gap;
a pair of parallel metallic strips located on the bottom side, wherein the pair of parallel metallic strips is configured to extend from the fourth edge towards the third edge, wherein the pair of parallel metallic strips has mirror geometry about the second axis; and
a coaxial feed port connected to the pair of parallel metallic strips at the fourth edge,
wherein inductance values of the first inductor and the second inductor and capacitance values of the first capacitor and the second capacitor are selected such that the folded dipole patch antenna resonates at a frequency of about 434 MHz upon application of an input signal at the coaxial feed port.

19. The folded dipole patch antenna of claim 18, wherein:
the first inductor has an inductance selected from the range of 0.4 nH to 0.8 nH;
the first capacitor has a capacitance selected from the range of 20 pF to 80 pF;
the second inductor has an inductance selected from the range of 0.4 nH to 0.8 nH; and
the second capacitor has a capacitance selected from the range of 20 pF to 80 pF.

20. The folded dipole patch antenna of claim 18, wherein:
the first gap is about 0.6 mm;
the second gap is about 1.0 mm;
each parallel strip has a length of about 7.0 mm,
each parallel strip has a width of about 4.4 mm; and
the third gap is about 1.0 mm.
